(19) United States
(12) Patent Application Publication
(10) Pub. No.: US 2025/0179115 A1
(43) Pub. Date: Jun. 5, 2025

(54) METHODS FOR PURIFYING BISPECIFIC ANTIBODIES

(71) Applicant: AstraZeneca AB, Södertälje (SE)

(72) Inventors: Matthew ASPELUND, Gaithersburg, MD (US); Kamiyar REZVANI, Gaithersburg, MD (US); Dominique WUDUNN, Gaithersburg, MD (US); Alan HUNTER, Gaithersburg, MD (US)

(21) Appl. No.: 18/844,113

(22) PCT Filed: Mar. 16, 2024
(86) PCT No.: PCT/EP2023/056721

§ 371 Date: Sep. 5, 2024

Related U.S. Application Data
(60) Provisional application No. 63/269,497, filed on Mar. 17, 2022.

(51) Int. Cl. C07K 1/22; B01D 15/38; C07K 16/00

(52) U.S. Cl. CPC C07K 1/22; B01D 15/3809; C07K 16/00; C07K 2317/31; C07K 2317/51; C07K 2317/515; C07K 2317/522; C07K 2317/55

(57) ABSTRACT

The present disclosure provides methods of purifying asymmetric bispecific antibodies using light chain affinity chromatography.

### 1. CROSS-REFERENCE TO RELATED APPLICATION

This application claims priority benefit to U.S. Provisional Patent Application No. 63/269,497, filed Mar. 17, 2022, which is incorporated by reference herein in its entirety for all purposes.

### 2. FIELD

The present disclosure relates generally to methods of purifying bispecific antibodies using light chain affinity chromatography.

### 3. BACKGROUND

Affinity chromatography is commonly employed for large scale manufacture and purification of numerous biologic therapeutics such as monoclonal antibodies. (K. M. Łącki, et al., Biotechnol. J. 15 (2020); G. R. Bolton et al., Biotechnol. Prog. 32 (2016) 1193-1202). Affinity chromatography provides process robustness due to the high specificity for products of interest and enables design of purification platforms which streamline process development and de-risk manufacturing (S. Sommerfeld, et al., Chem. Eng. Process. Process Intensif. 44 (2005)).

Various therapeutic formats, including multi-specific antibodies, fabs, and antibody fragments, utilize light chain (LC) affinity chromatography for product capture (G. Rodrigo, et al., Antibodies. 4 (2015) 259-277; J. Spooner, et al., Biotechnol. Bioeng. 112 (2015) 1472-1477; N. Fischer, et al., Nat. Commun. 6 (2015) 6113). Of interest here, asymmetric bispecific antibodies (bsAbs) are an attractive therapeutic modality due to their unique mechanisms of action and importantly their native antibody format which maintains favorable pharmacokinetic, stability, solubility, and immunogenicity profiles (A. F. Labrijn, et al., Nat. Rev. Drug Discov. 18 (2019) 585-608). However, these formats are historically plagued by the chain-association issue (C. Klein, et al., MAbs. 4 (2012) 653-663). Protein engineers have mediated the formation of heavy and light chain mis-pairing variants by employing various strategies such as steric and electrostatic steering, domain-crossover, common light chains, and kappa-lambda bodies, among many others (H. Lindhofer, et al., J. Immunol. 155 (1995) 219-25); J. B. B. Ridgway, et al., Protein Eng. Des. Sel. 9 (1996) 617-621; K. Gunasekaran, et al., J. Biol. Chem. 285 (2010) 19637-19646; J. H. Davis, et al., Protein Eng. Des. Sel. 23 (2010) 195-202; W. Schaefer, et al., Proc. Natl. Acad. Sci. 108 (2011) 11187-11192). Although chain pairing fidelity improves with these designs, mis-paired variants are still commonly observed after expression. Optimized expression procedures, in-vitro assembly, and high-throughput clone selection may also lessen the downstream burden, but often some level of these chain-mis-pairing impurities remain and must be controlled during downstream processing (A. F. Labrijn, et al., Proc. Natl. Acad. Sci. 110 (2013) 5145-5150; P. Strop, et al., J. Mol. Biol. 420 (2012) 204-219; G. Magistrelli, et al., MAbs. 9 (2017) 231-239; G. Giese, et al., Biotechnol. Prog. 34 (2018) 397-404; and C. Wang, et al., MAbs. 10 (2018) 1226-1235). Consequently, product related impurities remain a major hurdle towards developing commercial scale purification processes for asymmetric bsAbs and there is a need for additional scalable purification strategies.

### 4. SUMMARY

In some aspects, disclosed herein is a method of purifying an asymmetric bispecific antibody comprising: (a) contacting a composition comprising the bispecific antibody with a lambda light chain affinity matrix; (b) washing the affinity matrix to remove impurities; and (c) eluting the bispecific antibody with an elution buffer comprising between about 5 mM to about 45 mM of a salt.

In some aspects, the elution buffer further comprises an aggregation inhibitor. In some aspects, the aggregation inhibitor is arginine-HCl.

In some aspects, the concentration of the salt is about 20 mM. In some aspects, the halogen salt is sodium chloride, magnesium chloride, ammonium chloride, sodium acetate, sodium citrate, sodium phosphate, sodium sulfate, arginine-HCl, or histidine-HCl.

In some aspects, the pH of the elution buffer is between about 3 and about 4.5. In some aspects, the pH of the elution buffer is about 3.5.

In some aspects, the method further comprises contacting the asymmetric bispecific antibody with a kappa light chain affinity matrix and eluting the bound bispecific antibody. In some aspects, the contacting of the asymmetric bispecific antibody to a kappa light chain affinity matrix is performed prior to contacting the bispecific antibody with a lambda light chain affinity matrix. In some aspects, the eluent obtained following elution is directly passed over the second affinity matrix. In another aspect, the method is run in a closed system.

In some aspects, the kappa light chain affinity matrix and/or the lambda light chain affinity matrix is coupled to a solid support. In another aspect, the solid support is cross-linked agarose.

In some aspects, the bispecific antibody comprises a modified heavy chain and/or a modified light chain. In some aspects, the bispecific antibody comprises: (a) an Fab region comprising a modified heavy chain, wherein the CH1 region of the modified heavy chain comprises (i) a substitution of a native cysteine to a non-cysteine amino acid, and (ii) a substitution of a native non-cysteine amino acid to a cysteine amino acid; (b) a modified corresponding light chain, wherein the CL region of the modified light chain comprises (i) a substitution of a native cysteine to a non-cysteine amino acid, and (ii) a substitution of a native non-cysteine amino acid to a cysteine amino acid; (c) a second Fab region comprising a second heavy chain; and (d) a second corresponding light chain, wherein the modified heavy chain is directly linked to the corresponding modified light chain, and on a separate target binding arm, the second heavy chain is directly linked to the second corresponding light chain, and wherein the substituted cysteine of the modified heavy chain, resulting from the substitution of the native non-cysteine amino acid to the cysteine amino acid, and the substituted cysteine of the modified corresponding light chain, resulting from the substitution of the native non-cysteine amino acid to the cysteine amino acid, can form a disulfide bond. In some aspects, (a) the second heavy chain and second corresponding light chain do not comprise a substitution of a native non-cysteine amino acid to a cysteine amino acid and do not comprise a substitution of a native cysteine to a non-cysteine amino acid; and/or (b) the two light chains each comprise a VL domain and a CL domain, wherein the VL domains have different amino acid sequences and the CL domains have different amino acid sequences; and/or (c) the two heavy chains each comprise a VH domain, a CH1 domain and an Fc region, wherein the VH domains have different amino acid sequences, the CH1 domains have different amino acid sequences, and the Fc regions have different amino acid sequences, optionally wherein one light chain is a kappa light chain and one light chain is a lambda light chain. In some aspects, the two heavy chains form a heterodimer. In some aspects, the bispecific antibody specifically binds to two independent antigens or to two independent epitopes on the same antigen. In some aspects, the Fc region of either or both heavy chains comprises one or more modifications, optionally wherein the modifications facilitate heterodimerization of the heavy chains.

In some aspects, the methods result in the clearance of mis-paired species, removal of aggregates, and/or removal of low molecular weight impurities from the composition.

### 5. BRIEF DESCRIPTION OF THE FIGURES

FIG. 1 shows lambda light chains and corresponding heavy chains represented by yellow color and kappa light chains are represented by red color. Mis-paired species shown above would be observed in protein A product of captured cell culture harvest for each respective bsAb.

FIG. 2 shows chromatography elution profiles of bsAbs 1 & 2 on CaptureSelect Kappa XP, CaptureSelect Kappa XL, Capto L, and KappaSelect from pH gradient elution screening experiments. Absorbance at 280 nm (solid lines) is referenced by the left y-axis, and elution pH (dashed line) is referenced by the right y-axis.

FIG. 3 shows chromatography elution profiles of bsAbs 1 and 3 on lambda LC affinity media, from pH gradient elution screening experiments.

FIG. 4 shows HIC-HPLC analytical method absorbance traces of pH gradient elution screening products. Mis-pair composition of the split elution peaks are shown in comparison to CM purified by conventional methods, detailing mis-paired impurities present in the load material.

FIG. 5 shows the elution profiles of bsAb 2 on Kappa XP affinity media eluted with pH gradients and addition of (a) no modifier, (b) 250 mM sodium chloride, (c) 500 mM sodium citrate, (d) 500 mM MgCl, and (e) 500 mM L-Arginine.

FIG. 6 shows the elution profiles of bsAb 1 from CaptureSelect Lambda XP with pH 3.5 step elution conditions. The first peaks occur during the elution phase, and the second peaks occur during the strip phase.

### 6. DETAILED DESCRIPTION

In order that the present disclosure may be more readily understood, certain terms are first defined. As used in this application, except as otherwise expressly provided herein, each of the following terms shall have the meaning set forth below. Additional definitions are set forth throughout the application.

### 6.1 Terminology

Methods provided herein often are not limited to specific compositions or process steps, as such may vary.

The term “chromatography” refers to any kind of technique which separates a protein of interest (e.g., an antibody) from other molecules (e.g., contaminants) present in a mixture. Usually, the protein of interest is separated from other molecules (e.g., contaminants) as a result of differences in rates at which the individual molecules of the mixture migrate through a stationary medium under the influence of a moving phase, or in bind and elute processes. The term “matrix” or “chromatography matrix” or “chromatography media” are used interchangeably herein and refer to any kind of sorbent, resin or solid phase which in a separation process separates a protein of interest (e.g., an Fc region containing protein such as an immunoglobulin) from other molecules present in a mixture. Non-limiting examples include particulate, monolithic or fibrous resins as well as membranes that can be put in columns or cartridges. Examples of materials for forming the matrix include polysaccharides (such as agarose and cellulose); and other mechanically stable matrices such as silica (e.g. controlled pore glass), poly(styrenedivinyl)benzene, polyacrylamide, ceramic particles and derivatives of any of the above. Examples for typical matrix types suitable for the method of the present disclosure are cation exchange resins, affinity resins, anion exchange resins or mixed mode resins. A “ligand” is a functional group that is attached to the chromatography matrix and that determines the binding properties of the matrix. Examples of “ligands” include, but are not limited to, ion exchange groups, hydrophobic interaction groups, hydrophilic interaction groups, thiophilic interactions groups, metal affinity groups, affinity groups, bioaffinity groups, and mixed mode groups (combinations of the aforementioned). Some ligands that can be used herein include, but are not limited to, strong cation exchange groups, such as sulphopropyl, sulfonic acid; strong anion exchange groups, such as trimethylammonium chloride; weak cation exchange groups, such as carboxylic acid; weak anion exchange groups, such as N5N diethylamino or DEAE; hydrophobic interaction groups, such as phenyl, butyl, propyl, hexyl; and affinity groups, such as Protein A, Protein G, and Protein L.

The term “affinity chromatography” refers to a protein separation technique in which a protein of interest (e.g., an Fc region containing protein of interest or antibody) is specifically bound to a ligand which is specific for the protein of interest. Such a ligand is generally referred to as a biospecific ligand. In some aspects, the biospecific ligand (e.g., Protein A or a functional variant thereof) is covalently attached to a chromatography matrix material and is accessible to the protein of interest in solution as the solution contacts the chromatography matrix. The protein of interest generally retains its specific binding affinity for the biospecific ligand during the chromatographic steps, while other solutes and/or proteins in the mixture do not bind appreciably or specifically to the ligand. Binding of the protein of interest to the immobilized ligand allows contaminating proteins or protein impurities to be passed through the chromatography matrix while the protein of interest remains specifically bound to the immobilized ligand on the solid phase material. The specifically bound protein of interest is then removed in active form from the immobilized ligand under suitable conditions (e.g., low pH, high pH, high salt, competing ligand etc.), and passed through the chromatographic column with the elution buffer, free of the contaminating proteins or protein impurities that were earlier allowed to pass through the column. Any component can be used as a ligand for purifying its respective specific binding protein, e.g., antibody.

The terms “purifying,” “separating,” or “isolating,” as used interchangeably herein, refer to increasing the degree of purity of a protein of interest from a composition or sample comprising the protein of interest and one or more impurities. Typically, the degree of purity of the protein of interest is increased by removing (completely or partially) at least one impurity from the composition.

The term “buffer” as used herein, refers to a substance which, by its presence in solution, increases the amount of acid or alkali that must be added to cause unit change in pH. A buffered solution resists changes in pH by the action of its acid-base conjugate components. Buffered solutions for use with biological reagents are generally capable of maintaining a constant concentration of hydrogen ions such that the pH of the solution is within a physiological range. Traditional buffer components include, but are not limited to, organic and inorganic salts, acids and bases.

The term “chromatography column” or “column” in connection with chromatography as used herein, refers to a container, frequently in the form of a cylinder or a hollow pillar which is filled with the chromatography matrix or resin. The chromatography matrix or resin is the material which provides the physical and/or chemical properties that are employed for purification.

The terms “bind-and-elute” or “bind-and-elute mode” of purification refers to when the target protein of interest is retained on a column and impurities flow through the column. This process then involves specific elution of the protein of interest using different column conditions that interfere with the binding of the protein of interest to the chromatographic medium, usually a resin in a column.

The term “elution buffer” refers to a buffer that effectively dissociates protein: protein interactions without permanently affecting protein structure.

As used herein the term “contaminant” is used in its broadest sense to cover any undesired component or compound within a mixture. In cell cultures, cell lysates, or clarified bulk (e.g., clarified cell culture supernatant), contaminants include, for example, host cell nucleic acids (e.g., DNA) and host cell proteins present in a cell culture medium. Host cell contaminant proteins include, without limitation, those naturally or recombinantly produced by the host cell, as well as proteins related to or derived from the protein of interest (e.g., proteolytic fragments) and other process related contaminants. In certain aspects, the contaminant precipitate is separated from the cell culture using another means, such as centrifugation, sterile filtration, depth filtration and tangential flow filtration.

The term “loading buffer” refers to the buffer used to prepare and load a mixture or sample into the chromatography unit.

The term “chase buffer” refers to the buffer used subsequent to the loading buffer, in order to drive the mixture or sample through the chromatographic process.

The term “HMW Species” refers to any one or more unwanted proteins present in a mixture. High molecular weight species can include dimers, trimers, tetramers, or other multimers. These species are often considered product related impurities, and can either be covalently or non-covalently linked, and can also, for example, consist of misfolded monomers in which hydrophobic amino acid residues are exposed to a polar solvent, and can cause aggregation.

The term “LMW Species” refers to any one or more unwanted species present in a mixture. Low molecular weight species are often considered product related impurities, and can include clipped species, or half molecules for compounds intended to be dimeric (such as monoclonal antibodies).

The term “Host Cell Proteins” or HCP refers to the undesirable proteins generated by a host cell unrelated to the production of the intended protein of interest. Undesirable host cell proteins can be secreted into the upstream cell culture supernatant. Undesirable host cell proteins can also be released during cell lysis. The cells used for upstream cell culture require proteins for growth, transcription, and protein synthesis, and these unrelated proteins are undesirable in a final drug product.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

Where not expressly stated, and unless the context indicates otherwise, the term “antibody” includes monospecific, bispecific, or multi-specific antibodies, as well as a single chain antibody. In some aspects, the antibody is a bispecific antibody. The term “bispecific antibodies” refers to antibodies that bind to two different epitopes. The epitopes can be on the same target antigen or can be on different target antigens. The term “bispecific antibody” also refers to an antibody fragment, such as a Fab, that comprises both a kappa and lambda light chain.

The term “antibody fragment” refers to a portion of an intact antibody. An “antigen-binding fragment,” “antigen-binding domain,” or “antigen-binding region,” refers to a portion of an intact antibody that binds to an antigen. In the context of a bispecific antibody, an “antigen-binding fragment binds two antigens. An antigen-binding fragment can contain an antigen recognition site of an intact antibody (e.g., complementarity determining regions (CDRs) sufficient to specifically bind antigen). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

A “monoclonal” antibody or antigen-binding fragment thereof refers to a homogeneous antibody or antigen-binding fragment population involved in the highly specific binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that CDRs of the light and heavy chains are primarily responsible for the interaction and specificity of the antibody with antigen. In some aspects of the present disclosure, the variable region is a human variable region. In some aspects of the present disclosure, the variable region comprises rodent or murine CDRs and human framework regions (FRs). In particular aspects of the present disclosure, the variable region is a primate (e.g., non-human primate) variable region. In some aspects of the present disclosure, the variable region comprises rodent or murine CDRs and primate (e.g., non-human primate) framework regions (FRs).

As used herein, the term “heavy chain” or “HC” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects of the present disclosure, the heavy chain is a human heavy chain.

As used herein, the term “light chain” or “LC” when used in reference to an antibody can refer to any distinct type, e.g., kappa (κ) or lambda (λ) based on the amino acid sequence of the constant domains. Light chain amino acid sequences are well known in the art. In some aspects of the present disclosure, the light chain is a human light chain.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

As used in the present disclosure and claims, the singular forms “a,” “an,” and “the” include plural forms unless the context clearly dictates otherwise.

It is understood that wherever aspects of the present disclosure are described herein with the language “comprising,” otherwise analogous aspects described in terms of “consisting of” and/or “consisting essentially of” are also provided.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of 5% to 10% above and 5% to 10% below the value or range remain within the intended meaning of the recited value or range.

Any compositions or methods provided herein can be combined with one or more of any of the other compositions and methods provided herein.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

### 6.2 Bispecific Antibodies

In some aspects, the antibodies provided herein are monovalent bispecific antibodies (MBab). The monovalent bispecific antibody scaffolds described herein provide a superior platform for the generation of bispecific antibodies that fulfill all the benefits associated with bispecific antibodies while reducing the potential therapeutic risks mentioned above due to their monovalent nature. Furthermore, the MBabs provided herein are readily expressed, stable and are likely to have low immunogenicity. As used herein, the term “monovalent bispecific,” which may be abbreviated “MBab,” refers to bispecific antibodies (bsAb), where each arm can specifically bind to a different target antigen, and for a given pair of different target antigens (A and B), the MBab can bind to one of each. In certain aspects, monovalent bispecific antibodies can specifically bind to two independent antigens (or targets) or two independent epitopes on the same antigen. Typically, monovalent bispecific antibodies comprise two different variable regions. In some aspects, the binding affinity for the two independent antigens is about the same. In some aspects, the binding affinities for the two independent antigens are different. In some aspects, the binding affinity for two independent epitopes on the same antigen is about the same. In some aspects, the binding affinity for two independent epitopes on the same antigen is different. In still other aspects, each arm has the same specificity (e.g., binds the same, or an overlapping epitope) but binds with a different affinity. In some aspects, the affinities may differ by 3 fold or more. It may be particularly desirable to have one arm with higher affinity and one arm with lower affinity when combining variable regions from antibodies having different in vivo potencies to prevent the over or under dosing of one of the arms.

In certain aspects, an MBab binds the same epitopes or an overlapping epitopes on the same antigen (e.g. a receptor), with different affinities. In particular, same epitopes or overlapping epitopes, which are in close proximity when the antigen is dimerized. Such an antibody will have a dual characteristic depending on the relative concentration. For example, at high concentration, where the MBab concentration is saturating the antigen concentration, the high affinity binding domain will compete out the low affinity binding domain and little to no avidity effect will take place. That is the antibody will function primarily as a monovalent binding entity and little to no antigen cross-linking/signalling will take place. However, at low concentration avidity effects will come into play and the MBab can concurrently bind both binding sites, preferably on two antigen molecules, leading to antigen cross-linking/signaling. In this manner antigen signaling can be regulated by MBab concentration.

In certain aspects, a, MBab binds two different antigens (e.g. different receptors) where homodimerization of the antigens is undesirable and/or both antigens are present separately on non-targets cells/tissues and are present together on target cells/tissue. Such an antibody will bind with only one arm on non-target cells, this low avidity monovalent binding of only one arm of the MBab to non-target cells/tissues is insufficient to elicit homodimerization. In contrast the MBab can bind to both antigens on the target cells/tissue, binding to both antigens on the target cells simultaneously will result in a higher avidity bivalent binding that can enhance preferential binding to target cells and may enhance receptor dimerization.

In some aspects, the monovalent bispecific antibodies further comprise additional binding sites. The additional binding sites can be specific for one or both target antigens (A and B) of the monoclonal bispecific antibody (MBab) and/or can be specific for additional target antigens. In some aspects, one or more-single chain variable fragments (scFv) are added to the N- or C-terminus of one or both heavy chains and/or one or both light chains, where the one or more scFvs specifically bind to one or more additional target antigens. For example, a monovalent trispecific antibody can be generated by the addition of a scFv (specific for antigen C) to one chain (e.g., heavy or light) of a monovalent bispecific antibody (specific for antigens A and B). In this case, the antibody would be monovalent for antigens A, B, and C. If a scFv (specific for antigen C) is added to two chains (e.g. both heavy chains, both light chains, one heavy chain and one light chain), the trispecific antibody would be monovalent for antigens A and B and bivalent for antigen C. Any possible combination of additional binding sites is contemplated for the monovalent bispecific antibodies herein (see e.g., Dimasi et al. J. Mol. Biol. (2009) 393:672-692). It is contemplated that the binding affinity of the additional binding sites may be about the same as one or both arms of the MBab or may be different from one or both arms of the MBab. As described above, the relative affinities may be selected or tailored depending on the antigens and the intended use of the molecule.

Bispecific antibodies can be prepared as full length antibodies or antibody fragments (e.g. F(ab′)2 bispecific antibodies). Traditional production of full length bispecific antibodies is based on the co-expression of two immunoglobulin heavy chain-light chain pairs, where the two chains have different specificities. Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields can be low.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods. This method, however, typically requires the use of non-human proteins, which can carry high immunogenicity potential. Further, the antibody fragments sometimes have little or no effector function and a short half-life.

Bispecific antibodies can be prepared using chemical linkage. In one procedure intact antibodies are proteolytically cleaved to generate F(ab′)2 fragments. These fragments are reduced in the presence of the dithiol complexing agent, sodium arsenite, to stabilize vicinal dithiols and prevent intermolecular disulphide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. This method, however, often leads to poor yield, is difficult to control, and the products can carry high immunogenicity potential. Further, the antibody fragments sometimes have little or no effector function and a short half-life.

Fab′-SH fragments can be directly recovered from E. coli, which can be chemically coupled to form bispecific antibodies. A fully humanized bispecific antibody F(ab′)2 molecule may be created by secreting each Fab′ fragment separately from E. coli and subjecting to directed chemical coupling in vitro to form the bispecific antibody. This method, however, often leads to poor yield and is difficult to control. Further, the antibody fragments sometimes have little or no effector function and a short half-life.

Bispecific antibodies may also be produced using leucine zippers. The leucine zipper peptides from the Fos and Jun proteins are linked to the Fab′ portions of two different antibodies by gene fusion. The antibody homodimers are reduced at the hinge region to form monomers and then re-oxidized to form the antibody heterodimers. This method can also be utilized for the production of antibody homodimers. The “diabody” technology described has provided an additional mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers is also known in the art. This method, however, often leads to poor yield, is difficult to control, and the products can carry high immunogenicity potential. Further, the antibody fragments sometimes have little or no effector function and a short half-life.

Bispecific antibodies may also be produced using heavy chain heterodimerization methods. Such methods include “knob in hole” and strand-exchanged engineered domain (SEED) methods, and those which alter the charge polarity across the Fc dimer interface. Such methods are described in further detail herein and in e.g., U.S. Pat. No. 7,183,076; Merchant et al. (1998) Nat. Biotech 16:677-681; Ridgway et al. (1996) Protein Engineering 9:617-621; Davis et al. (2010) Prot. Eng. Design & Selection 23:195-202; WO 2007/110205; WO 2007/147901; Gunasekaran et al. (2010) JBC 285:19637-46. In these methods, the interface between a pair of antibody molecules may be engineered to maximize the percentage of heterodimers which are recovered from recombinant cell culture. In the “knob in hole” method, a “protrusion” is generated by replacing one or more small amino acid side chains from the interface of the first antibody molecule with larger side chains (e.g. tyrosine or tryptophan). Compensatory “cavities” of identical or similar size to the large side chain(s) are created on the interface of the second antibody molecule by replacing amino acid having large side chains with amino acids having smaller ones (e.g. alanine or threonine). This provides a mechanism for increasing the yield of the heterodimer over other unwanted end-products such as homodimers. In the SEED method, Fc homodimers are converted into heterodimers by interdigitating beta-strand segments of human IgG and IgA CH3 domains. These derivatives of human IgG and IgA CH3 domains create complementary human SEED CH3 heterodimers that are composed of alternating segments of human IgA and IgG CH3 sequences. The resulting pair of SEED CH3 domains preferentially associates to form heterodimers when expressed in mammalian cells. Other methods include introducing modifications which alter the charge polarity across the Fc dimer interface such that co-expression of electrostatically matched Fc regions results in heterodimerization. These methods improve heavy chain heterodimerization, but do not address the light-heavy chain mispairings formed during bispecific antibody formation. In some cases use of a common light chain can decrease the number of possible mispairings, as described in WO 98/50431, but often results in the loss or reduction of binding specificity and/or affinity.

Duel specific antibodies are another type of bispecific antibody that can be produced (see e.g. Bostrum et al. (2009) Science 323:1610-1614). Such antibodies can be produced by generating variants with mutations in the light chain (LC) complementarity determining regions (CDR) such that they can bind a new antigen target while maintaining binding specificity for its native target antigen. The antigen binding sites often overlap, however, preventing the antibody from binding both antigens at the same time. Additionally, these antibodies are difficult to generate and may not possess the desired affinities for each of the two antigens.

The modified polypeptides provided herein can be useful for the generation of bispecific antibodies and overcome the limitations and technical difficulties noted above. In some aspects, one heavy chain and one light chain within an antibody are modified whereby a native cysteine is substituted by a non-cysteine amino acid, and a native non-cysteine amino acid is substituted by a cysteine amino acid. Such modifications provided herein are generated in the HC and LC domains and result in the relocation of an HC-LC interchain disulphide bridge. When generating a bispecific antibody from four separate polypeptides, for example, where the modified arm has a binding specificity for one target and the unmodified arm has a binding specificity for a different target, the four polypeptides will assemble such that the modified heavy chain properly hybridizes with the modified light chain and the unmodified heavy chain properly hybridizes with the unmodified light chain. As used herein, the term “unmodified” refers to heavy and light chains that do not contain the HC-LC modifications introduced for the relocation of cysteines and/or disulphide bridges, as described herein. Such “unmodified” heavy and light chains may comprise other modifications, such as, for example, heterodimerization modifications in the CH2 and/or CH3 regions described herein and/or known in the art. The HC-LC modifications provided herein can be combined with further modifications of the heavy chain, particularly in the CH2 and/or CH3 regions to ensure proper heavy chain heterodimerization and/or to enhance purification of the a heavy chain heterodimer and are described in detail below.

The bispecific antibodies of the invention can be asymmetric, containing one kappa light chain and one lambda light chain. They can also contain HC and/or LC such as those described in U.S. Pat. No. 9,527,927, which is herein incorporated by reference.

### 6.3 Methods of the Invention

The present invention evaluates the capability of commercially available LC affinity chromatography media to separate multiple product-related impurities of selected asymmetric bsAb constructs comprised of four unique polypeptide chains (Y. Mazor, et al., MAbs. 7 (2015) 377-389). Currently, several scalable chromatography media options with specificity for kappa and lambda light chains are commercially available. Protein L media provide specificity to the variable region of certain kappa light chain subtypes while other kappa and lambda LC affinity media provide specificity for the constant regions of kappa and lambda light chains (M. Graille, et al., Structure. 9 (2001) 679-687). Apart from protein L, these LC affinity media employ camelid-antibody-based ligand technology (C. Hamers-Casterman, et al., Nature. 363 (1993) 446-448; J. T. Detmers, et al., Bioprocess Int. 8 (2010) 50-54; M. Zandian, et al., J. Chromatogr. A. 1216 (2009) 5548-5556; and T. M. Pabst, et al., Biotechnol. J. 12 (2017) 1600357). Previous literature describes the development of one example of these chromatography media, Lambdafabselect, in depth (N. Eifler, et al., Biotechnol. Prog. 30 (2014) 1311-1318). Two mechanisms of separation by LC affinity chromatography are described herein, one where impurities missing a light chain of the class as the affinity media specificity will flow-through the column, and the second where impurity species possessing more than one light chain moiety of the same class as the resin specificity will be more strongly retained compared to the target heterodimer product.

An increasing body of literature describes the application of affinity chromatography media to separate product-related impurities based on binding strength. Notable examples include exploiting pH during elution to separate monoclonal antibody monomer from aggregate, polyclonal human IgG sub-populations, and Fc-binding-ablated bsAbs on Protein A and G affinity media (C. Andrade, et al., Biotechnol. Prog. 35 (2019) e2720; J. Weinberg, et al., Biotechnol. Bioeng. 114 (2017) 1803-1812; A. D. Tustian, et al., MAbs. 8 (2016) 828-838; and R. Ollier, et al., MAbs. 11 (2019) 1464-1478). Additionally, separation of bsAb mis-paired variants containing two light chains of the same class as the resin specificity has been demonstrated for kappa light chains using protein L and KappaSelect media (C. Chen, et al., MAbs. 11 (2019) 632-638; S. W. Chen, et al., MAbs. 12 (2020); and T. Qin, etl al., Protein Expr. Purif. 171 (2020)). Data from Tustian et al. for protein A, and Chen et al. for protein L media also outline the benefit of fine-tuning avidity-based selectivity with conductivity and different modifiers during elution rather than elution pH alone (A. D. Tustian, et al., MAbs. 8 (2016) 828-838; and S. W. Chen, et al., MAbs. 12 (2020)). These findings have increased the understanding of the development space for protein A & L mediated avidity separations, but increased understanding for other kappa and lambda LC affinity media is desired owing to their specificity to constant regions of light chains. LC affinity capture processes, particularly those which bind constant regions of light chains, are attractive because they provide platformable approaches towards purification development for numerous emerging antibody-based therapeutic formats, such as chain-mis-pairing bsAbs.

Therefore, the invention pertains to a method for purifying a protein from solution comprising contacting a composition comprising the bispecific antibody with a lambda light chain affinity matrix; (b) washing the affinity matrix to remove impurities; and (c) eluting the bispecific antibody with an elution buffer comprising between about 5 mM to about 45 mM of a halogen salt. Varioius lambda light chain affinity matrices are well known in the art and suitable for use in the invention. Non-limiting examples of commercially available LC resins include Capture Select™, LambdaFabSelect, and Capto™ L.

Binding of the bsAb to the LC matrix typically is achieved by column chromatography. That is, the LC matrix is formed into a column, a biochemical mixture containing a bsAb is flowed through the column, followed by washing of the column by flowing through the column one or more wash solutions, followed by elution of the protein of interest from the column by flowing through the column an elution buffer.

Alternatively, binding of the bsAb to the LC matrix can be achieved by batch treatment, in which the biochemical mixtures containing the bsAb is incubated with the LC matrix in a vessel to allow for binding of the bsAb to the LC matrix, the solid phase medium is removed from the vessel (e.g., by centrifugation), the solid phase medium is washed to remove impurities and again recovered (e.g., by centrifugation) and the bsAb is eluted from the solid phase medium.

In yet another aspect, a combination of batch treatment and column chromatography can be used. For example, the initial binding of the bsAb to the LC matrix can be achieved by batch treatment and then the solid phase medium can be packed into a column, following by washing of the column and elution of the bsAb from the column.

Most affinity purification procedures involving protein: ligand interactions use binding buffers at physiologic pH and ionic strength, such as phosphate buffered saline (PBS). Once the binding interaction occurs, the support is washed with additional buffer to remove nonbound components of the sample. Nonspecific (e.g., simple ionic) binding interactions can be minimized by adding low levels of detergent or by moderate adjustments to salt concentration in the binding and/or wash buffer.

Halogen salts, including salts containing chlorine (Cl) and bromine (Br), in particular halogen salts containing alkali or alkaline earth metals, including sodium, potassium, calcium and magnesium are referred to non-buffer salts. In one espect, the elution buffer comprises a halogen salt (e.g, containing Cl or Br). In another aspect, the non-buffer salt is a halogen salt containing sodium (Na), potassium (K), calcium (Ca) or magnesium (Mg). In one aspect, the elution buffer comprises Na or Mg. In another aspect, the halogen salt is NaCl or MgCl₂.

The present invention demonstrates that low levels of halogen salts are effective at purifying bsAbs from the affinity matrix. It has been known that high levels, for example 100 mM, is effective for removing impurities, but these high levels have a negative effect on bsAb yield. Therefore, the concentration of halogen salt in the elution buffer is between about 5 mM and 45 mM. In some aspects, the concentration of halogen salt is about 5 mM, about 10 mM, about 15 mM, about 20 mM, about 25 mM, about 30 mM, about 35 mM, about 40 mM or about 45 mM.

The elution buffer can also comprise an aggregation inhibitor. In some aspects, the aggregation inhibitor is arginine or an arginine derivative. The arginine which can be used in can be the natural amino acid arginine (e.g., L-arginine), D-arginine or an arginine derivative. Non-limiting examples of arginine derivatives include acylated arginine, such as acetyl arginine and N-alpha-butyroyl-arginine, agmatine, arginic acid and N-alpha-pyvaloyl arginine. The arginine or arginine derivative can be used in the form of an acid addition salt. Examples of the acid which can form an acid addition salt include hydrochloric acid and the like.

The pH of the elution buffer of the invention typically acidic. In one aspect, the pH of the elution buffer is between about 3 and about 4.5, depending on the properties of the bsAb. In some aspects, the pH is about 3, about 3.5, about 4, or about 4.5. In some aspects, the elution buffer has a pH of 3.5.

The method of the invention can be performed in a multi-column chromatography system, comprising at least a first and second chromatography matrix. The term “multi-column chromatography system” means a system of a total of two or more separate, interconnected, or switching chromatography columns and/or chromatographic membranes. Additional examples of multi-column chromatography systems are known in the art.

In one aspect, the second chromatography matrix is a kappa light chain affinity matrix. Commercially available kappa light chain affinity matrices are known in the art, non-limiting examples include CaptureSelect™ LC-kappa. In one aspect, the first and second chromatography steps can be performed in any order.

The chromatography column(s) and/or chromatographic membrane(s) present in a multi-column chromatography system can be connected or moved with respect to each other by a switching mechanism (e.g., a column-switching mechanism). The column-switching events can be triggered by the detection of a level of the protein to be purified detected by UV absorbance corresponding to a certain level of protein in the fluid passing through the multi-column chromatography system (e.g., the input into and/or eluate from one or more of the chromatography column(s) and/or chromatographic membranes in the system), a specific volume of liquid (e.g., buffer), or specific time elapsed. Column switching generally means a mechanism by which at least two different chromatography columns and/or chromatographic membranes in a multi-column chromatography system (e.g., two or more different chromatography columns and/or chromatographic membranes present in a system are allowed to pass through a different step (e.g., equilibration, loading, eluting, or washing) at substantially the same time during at least part of the process.

The method of the invention can be run in continuous mode. In other words, the method of the invention can be a continuous method for purifying a bsAb from solution. The term “continuous method” or “method in a continuous mode” means a method which continuously feeds fluid through at least a part of the system.

The following examples are offered by way of illustration and not by way of limitation.

### 7. EXAMPLES

The examples in this Section are offered by way of illustration, and not by way of limitation.

Protein sample preparation. All bsAbs were produced at AstraZeneca from a Chinese Hamster Ovary (CHO) cell expression system. Cell culture supernatant was harvested by centrifugation and depth filtration to produce conditioned cell culture media (CM). CM was then processed by standard protein A or lambda light chain affinity chromatography methods to produce purified material containing relevant mis-paired species for further experimentation.

Stationary phases, chromatography equipment, and reagents. KappaSelect, Capto L, and LambdaFabSelect LC affinity media were purchased from Cytiva, formerly GE Healthcare, (Uppsala Sweden). CaptureSelect Kappa XL and XP, and CaptureSelect Lambda XP LC affinity media were obtained from Thermo Fisher Scientific (Waltham, MA). Chromatography media attributes were obtained through publicly visible vendor literature, Table 1. Chromatography experiments were completed on an AKTA Avant 25 system controlled by Unicorn 7.0 software from Cytiva. Chromatography media were packed into Omnifit column hardware (0.66 cm I.D.) supplied by Cole-Parmer (Vernon Hills, IL) or Vantage column hardware (1.15 cm I.D.) supplied by MilliporeSigma (Burlington, MA). All chemicals used for buffer preparation were obtained from JT Baker (Phillipsburg, NJ).

### Table 1

### Summary of Light Chain Affinity Media Attributes

TABLE 1
Summary of Light Chain Affinity Media Attributes

| | | | | Average | | | |
|---|---|---|---|---|---|---|---|
| | | | | particle | Ligand | | |
| Affinity | | Speci- | Ligand | diameter | density | Coupling | DBC_{10%}^{c} |
| media | Manufacturer | ficity^{a} | type | (μm) | (mg/mL) | chemistry | (mg/mL) |
| KappaSelect | Cytiva | cκ | Camelid | 75 | 5 | Amide linkage | 35 |
| CaptureSelect | ThermoFisher | cκ | Camelid | 65 | —^{b} | Aldehyde coupling | 33 |
| Kappa XL | Scientific | | | | | | |
| CaptureSelect | ThermoFisher | cκ | Camelid | 65 | —^{b} | Epoxide coupling | 45 |
| Kappa XP | Scientific | | | | | | |
| Capto L | Cytiva | vκ | Recombinant | 85 | —^{b} | Epoxy activation | 46 |
| | | | protein L | | | | |
| LambdaFab | Cytiva | cλ | Camelid | 75 | 7 | Amide linkage | 32 |
| Select | | | | | | | |
| CaptureSelect | ThermoFisher | cλ | Camelid | 65 | —^{b} | Epoxide coupling | 39 |
| Lambda XP | Scientific | | | | | | |

^{a}Region of antibody light chains which the chromatography media has high affinity and specificity for
^{b}Undisclosed by manufacturer
^{c}Value measured with purified bsAb load at 4 minute residence time

^{a} Region of antibody light chains which the chromatography media has high affinity and specificity for
^{b} Undisclosed by manufacturer
^{c} Value measured with purified bsAb load at 4 minute residence time

Protein concentration determination. Product concentration in CM were quantified by protein A and lambda LC affinity HPLC techniques using a standard curve generated from purified bsAb. CaptureSelect Protein A and CaptureSelect Lambda analytical columns were purchased from Thermo Fisher Scientific (Waltham, MA). Product concentration in purified samples were determined by absorbance at 280 nanometer wavelength with a Nanodrop 2000c spectrophotometer purchased from Thermo Fisher Scientific.

Analytical instrumentation, materials, and methods. Mis-paired, aggregate, and fragment species content were quantified by multiple orthogonal analytical techniques. High-performance size-exclusion chromatography (HP-SEC) was performed using a GS3000SWXL column (7.9×300 mm) purchased from Tosoh Bioscience (Tokyo, Japan). High-performance hydrophobic interaction chromatography (HIC-HPLC) was performed using a mAb-Pac HIC 10 analytical column purchased from Thermo Fisher Scientific. HPLC-based methods were operated on Agilent 1260 HPLC systems (Palo Alto, CA). Non-reduced capillary gel electrophoresis (NR-CGE) was performed on a LabChip GXII system using the HT Protein Express reagent kit from PerkinElmer (Waltham, MA).

Dynamic binding capacity determination. Approximate dynamic binding capacities at 10% product break-through (DBC_{10%}) of LC affinity chromatography media were obtained by overloading the media with purified bsAb to generate product breakthrough curves. LC affinity media were loaded to 100 mg of bsAb per mL packed bed at a 4-minute residence time. Approximate DBC_{10%} values were calculated from the volume at which the absorbance of the product break-through during load, measured by the AKTA UV detector, measured 10% of the absorbance of the load material bypassed the column. System dead volumes were subtracted from the volume at 10% breakthrough and this volume was then converted to the protein load challenge. Prior evaluation demonstrates excellent linearity between 280 nm absorbance measurements by the AKTA UV detector and protein concentration up to the maximum protein concentration of the load material used.

Identification of mis-paired and product related impurity species. The HIC-HPLC assay was used to quantify mis-pair levels in chromatography product and/or load material, HP-SEC was used to quantify aggregate and monomer, and NR-CGE was used to quantify half antibody, LC dimer and free LC. HIC-HPLC purity was also presented as a general product quality metric, but notably does not account for aggregate content.

pH gradient elution screening. Chromatography was executed at 4-minute residence time and LC affinity media were load to 10 mg bsAb/mL packed bed. Products were eluted with a 20 column volume (CV) 50 mM sodium citrate/citric acid, pH 6.0-2.5 linear gradient. Elution peaks were fractionated, and individual peaks pooled. Protein concentrations were determined and product pools were subsequently pH neutralized, and analyzed for aggregate, fragment, and mis-pair content.

Resolution (Rs) between elution peaks was supplied by Unicorn software through its automated peak detection and from the equation:

$$R s = 1.18 \frac{{t}_{R 1} - {t}_{R 2}}{{w}_{h 1} + {w}_{h 2}}$$

where t_{R1} is peak retention time and wₕ is peak width at half height.

Approximate elution pH of product peaks were calculated through pH measurements by the AKTA pH detector at peak maximum absorbance. System dead volumes were subtracted from the volume at peak maximum, and the corresponding pH was recorded. The pH electrode was calibrated prior to each run. Approximate pH values obtained agreed qualitatively with overlays of elution peak profiles across experiments.

### Purification of Asymmetric Bispecific Antibody Heterodimers

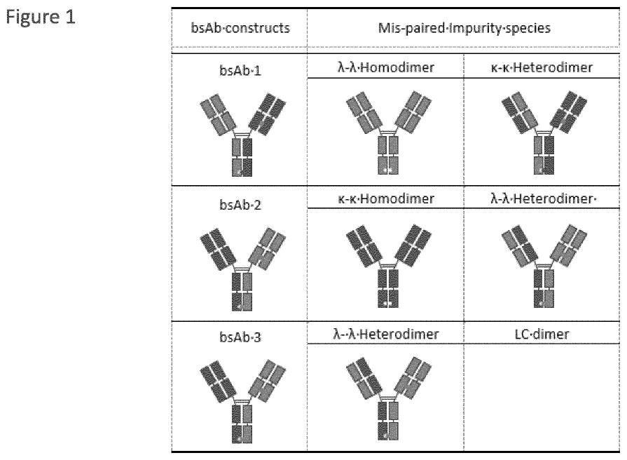

Binding avidity can be leveraged to separate protein species with different numbers of binding motifs on affinity media, also described as binding valency. Generally, for affinity chromatography media such as protein A, G, L, and camelid-antibody derived media, separation can be achieved by applying a descending pH elution gradient. Commercially available LC affinity media were probed for separation of asymmetric bispecific antibody heterodimers containing one lambda and one kappa light chain from mis-paired species containing two light chains of the same class as the stationary phase specificity. pH gradient elution experiments were first performed on selected commercially available kappa and lambda LC affinity chromatography media to evaluate the “baseline” selectivity based on product binding valency. Cell culture harvest material containing bsAb 1 or 2 were used to evaluate separation of kappa-kappa mis-paired species by kappa LC affinity chromatography, and cell culture harvest material containing bsAb 1 or 3 were used for separation of lambda-lambda mis-paired species by lambda affinity chromatography, FIG. 1.

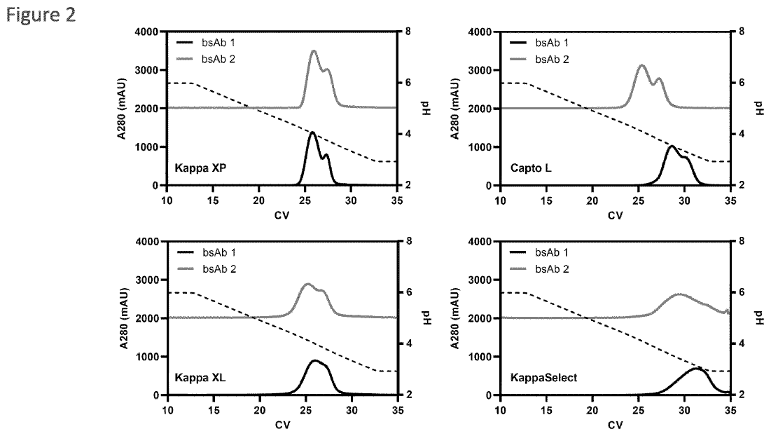

Results from the initial screen illustrated in FIGS. 2, 3, and 4 outline the separation of mis-paired impurities in the form of split elution peaks. Among kappa LC affinity media tested, CaptureSelect Kappa XP provided greatest resolution for bsAb 1, and Capto L provided greatest resolution for bsAb 2, Table 2.

TABLE 2
LC affinity pH gradient process performance summary

| | Resolution^{a} (Rs) | | |
|---|---|---|---|
| Media | bsAb 1 | bsAb 2 | bsAb 3 |
| KappaSelect | 0 | 0 | —^{b} |
| CaputreSelect Kappa XL | 0.35 | 0.47 | —^{b} |
| CaptureSelect Kappa XP | 0.64 | 0.58 | —^{b} |
| Capto L | 0.42 | 0.62 | —^{b} |
| LambdaFabSelect | 0.73 | —^{b} | 0.36 |
| CaptureSelect Lambda | 0.79 | —^{b} | 0.40 |
| XP | | | |

^{a}Resolution between 1^{st} and 2^{nd} elution peaks on chromatogram, as calculated by Unicorn software.
^{b}Not tested.

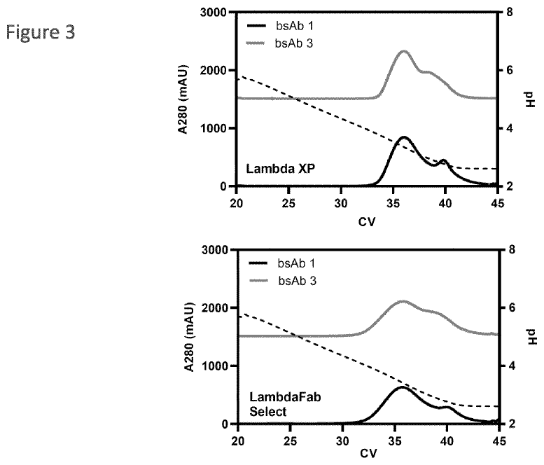

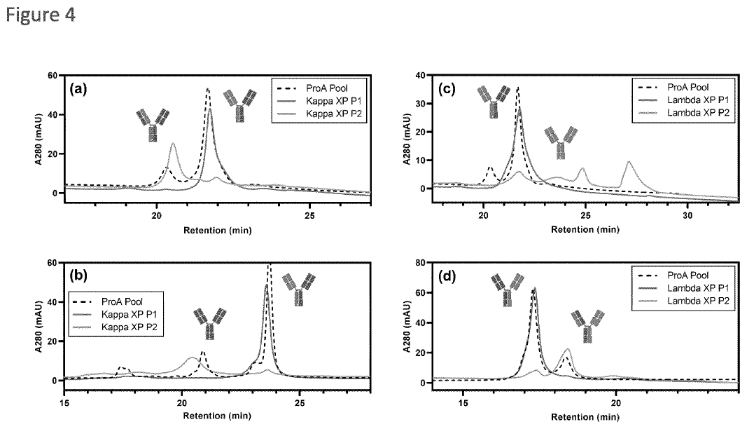

The difference in chromatography performance for the two bsAbs may be attributed to protein L's specificity for the variable region of kappa light chains. In this case bsAb 2 is eluted earlier in the pH gradient from protein L compared to bsAb 1. As expected due to their specificity for constant regions of kappa light chains, CaptureSelect Kappa XP, CaptureSelect Kappa XL, and KappaSelect provided consistent retention between bsAbs 1 and 2, with some disproportion in peak 1 and 2 sizes for the different bsAbs, FIG. 2. This disproportion can be attributed to the different levels of mis-paired and other product-related impurities present in CM material for bsAb 1 and 2. Among lambda LC affinity media tested, CaptureSelect Lambda XP media provided more distinct peak separation than LambdaFabSelect for both bsAbs 1 and 3, FIG. 3. For product quality testing, elution fractions for peaks 1 and 2 were pooled and differentiated as close to the peak inflections as possible. HIC-HPLC product quality data confirms that the distinct peaks observed are predominantly due to heterodimer and light chain mis-paired variant separation, FIG. 4. The second elution peaks were also enriched in aggregate and LC dimer for all bsAbs tested, Table 3. Unexpectedly, free light chains (by NR-CGE) were enriched in the second peaks for all experiments, and kappa LC affinity media clearly provided enrichment of half antibody (by NR-CGE) in the second elution peak for bsAb 2, Table 3. Enrichment of half antibody in the second elution peak however was not as clearly observed on Lambda affinity media for bsAb 1 or 3.

TABLE 3
Impurity separation by pH gradient elution

| | | | | | Impurity content | | |
|---|---|---|---|---|---|---|---|
| | | | Impurity content | | by NR-CGE (%) | | |
| | bsAb | Elution | by SEC (%) | | Half | LC | Free |
| Media | construct | peak | Monomer | Aggregate | antibody | dimer | LC |
| CaptureSelect | bsAb 1 | 1 | 75.0 | 8.8 | 0 | 0.8 | 0.8 |
| Kappa XP | | 2 | 70.8 | 20.0 | 0 | 4.2 | 9.7 |
| | bsAb 2 | 1 | 89.9 | 1.3 | 2.0 | 0.7 | 1.3 |
| | | 2 | 63.4 | 10.5 | 19.9 | 7.1 | 4 |
| Capto L | bsAb 1 | 1 | 84.8 | 9.6 | 0 | 0.8 | 0.6 |
| | | 2 | 62.8 | 20.6 | 0 | 11.1 | 10.6 |
| | bsAb 2 | 1 | 89.3 | 0.7 | 3.0 | 0 | 0.9 |
| | | 2 | 68.5 | 9.5 | 13 | 5.1 | 2.6 |
| LambdaFabSelect | bsAb 1 | 1 | 83.2 | 9.3 | 1.0 | 1.0 | 0.8 |
| | | 2 | 27.9 | 27.4 | 0.6 | 13.1 | 69.7 |
| | bsAb 3 | 1 | 90.7 | 2.6 | 0.5 | 1.9 | 1.5 |
| | | 2 | 59.1 | 5.8 | 1.1 | 26.5 | 22.0 |
| CaptureSelect | bsAb 1 | 1 | 85.1 | 5.8 | 1.0 | 1.6 | 1.0 |
| Lambda XP | | 2 | 29.4 | 31.2 | 0.3 | 20.3 | 59.9 |
| | bsAb 3 | 1 | 94.4 | 1.6 | 0.3 | 1.0 | 1.1 |
| | | 2 | 59.0 | 4.0 | 1.2 | 23.9 | 26.6 |

Differences in peak resolution observed from the pH gradient screening may be credited to specific attributes of the different chromatography matrices. These attributes include resin particle diameters, pore sizes, and pore morphologies which can provide more favorable mass transfer properties, and ligand densities and architectures which together can provide higher binding capacities and increased availability for multi-binding events. In terms of base matrix-specific effects, CaptureSelect Kappa XL and Kappa XP media notably both use the same agarose matrix but display markedly different peak resolution for our bsAb heterodimers and mis-pairs, FIG. 2. Comparatively, LambdaFabSelect and Lambda XP use distinct agarose base matrices, notably with the average particle diameter of Lambda XP slightly smaller, Table 1. Product literature reveals that Kappa XP uses epoxide coupling while Kappa XL uses aldehyde coupling chemistry for ligand attachment, and Lambda XP similarly uses epoxide coupling while LambdaFabSelect uses an amide linkage. Epoxide-chemistry-mediated single-point ligand coupling was hypothesized to be a major contributor towards this increase in observed separation with CaptureSelect Kappa XP and Lambda XP media, compared to multi-point ligand attachment with CaptureSelect Kappa XL and LambdaFabSelect. Single point coupling, compared to multi-point, may provide more uniform ligand coupling, increased flexibility, and more favorable orientations to provide enhanced ligand exposure and opportunity for multi-binding events. Changes to ligand coupling may also impact binding and displacement kinetics as described by Weinberg et al., (Biotechnol. Bioeng. 114 (2017) 1803-1812). These differences may affect resolution due to the impact they can have on protein migration through the column. Deeper investigation into ligand and attachment specific effects, and potential contribution of binding and displacement kinetics could provide a clearer picture on the differences in peak resolution observed here.

Subsequently, the impact of various elution modifiers to bsAb mis-pair separation on Kappa XP and Lambda XP media due to their ability to provide greatest peak resolutions in our screening experiments was evaluated. Previous work outlines the ability to increase resolution of avidity-based affinity separations by incorporating various salts in the elution buffer and/or controlling elution conductivity (C. Chen, et al., MAbs. 11 (2019) 632-638; S. W. Chen, et al., MAbs. 12 (2020; and A. D. Tustian, et al., MAbs. 8 (2016) 828-838). For Kappa XP, pH gradient experiments were performed with the addition of 0.5M sodium citrate (NaCitrate), magnesium chloride (MgCl), or L-arginine-HCl (arginine), or 0.25M sodium chloride (NaCl) salts. Protein A-purified bsAb 2 load material was used for these experiments and results were compared to a control experiment with no salt addition. A lower 0.25M NaCl concentration was used due to substantial precipitation observed at higher concentrations. High concentrations of these salts were needed to maximize the separation as lower concentrations provided decreased resolution between heterodimer and mis-paired impurities (data not shown). Additionally, the elution gradient with MgCl was modified to pH 4.3-3.0 due to solution buffering characteristics and arginine addition similarly produced a slight impact to pH gradient linearity.

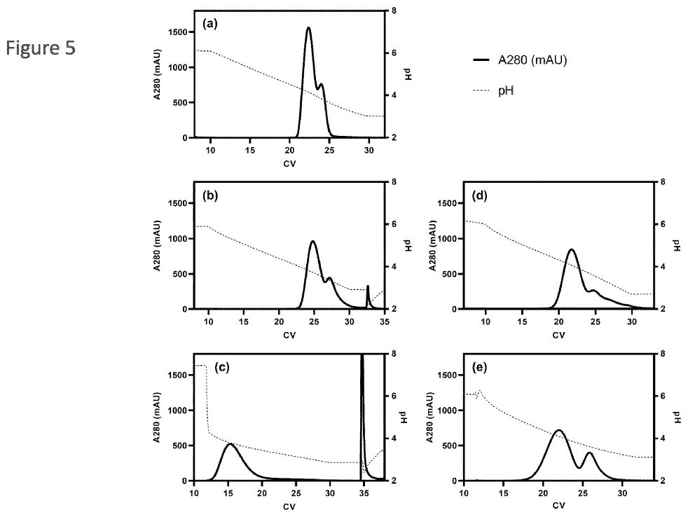

Elution peak broadening was observed with all modifiers and increased impurity peak separation with MgCl₂ and arginine, FIG. 5. MgCl₂ addition provided the greatest increase to resolution with surprising baseline separation. Increases to resolution with MgCl₂ and arginine additions appear to be due to preferential strengthening of the kappa-kappa mis-pair and half antibody binding to the media. The baseline pH gradient condition with no modifier addition produced elution of the second peak at approximately pH 3.9, while for MgCl₂ the second peak elution did not occur even by pH 2.8, and for arginine elution occurred at pH 3.56, Table 4. Process performance and product quality with the addition of these modifiers on Kappa XP media is provided in Table 4, and notably shows that NaCl, MgCl and arginine additions produced large increases in aggregate in the heterodimer peak elution pool. Separation of half antibody and co-elution with kappa-kappa mis-paired species was maintained with addition of the modifiers, while impact of light chain impurities was not evaluated due to absence of light chain impurities in the protein A purified material. Despite the high degree of separation for mis-paired impurities observed with MgCl₂, difficulties around high modifier concentrations, protein stability, large elution pool volume, and low product recovery necessitate further optimization to possibly apply this method towards large scale protein purification.

TABLE 4
CaptureSelect Kappa XP Elution Modifier Process Performance

| | | Peak 1 | Peak 1 | | | |
|---|---|---|---|---|---|---|
| | | purity | aggregate | | | |
| | Step | by HIC- | by | Peak 1 | Peak 1 | Peak 2 |
| Elution | Yield | HPLC | HP-SEC | volume | elution | elution |
| Condition | (%) | (%) | (%) | (CV) | pH | pH |
| Load | — | 74.7 | 3.8 | — | — | — |
| No modifier | 70 | 93.4 | 1.7 | 2.7 | 4.15 | 3.89 |
| NaCitrate | 62 | 95.7 | 2.7 | 4.1 | 4.10 | 3.66 |
| NaCl | 61 | 93.6 | 43.8 | 3.4 | 3.91 | 3.56 |
| MgCl₂ | 58 | 96 | 18.2 | 8.0 | 3.74 | —^{a} |
| Arginine | 69 | 93.8 | 9.2 | 5.6 | 4.08 | 3.56 |

^{a}Elution pH unable to be determined with experiment conditions.

For both Lambda XP and LambdaFabSelect media, it was expected that a much larger impact on elution performance in presence of salts would occur compared to the Kappa XP results. From initial screening, incomplete product elution at pH 2.8 was observed with addition of any tested salt at 100 mM concentration on LambdaFabSelect. Additionally, for pH gradient experiments, a base buffer of 50 mM sodium citrate likely contributed more substantially to changes in binding interactions between bsAb and lambda affinity media. Step elution experiments at pH 3.5 were performed using a 25 mM sodium acetate elution buffer to minimize baseline buffer conductivity, and with the addition of 20 mM NaCl, arginine, or MgCl₂ to evaluate impact to product binding strength with modifiers. A control step elution experiment at pH 3.5 was performed with no modifier.

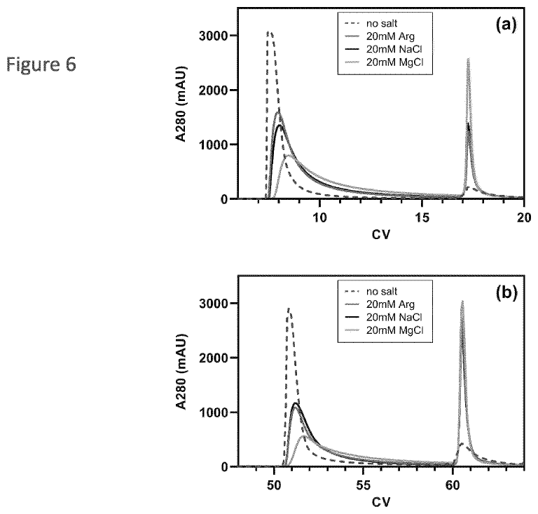

As seen in FIGS. 6a and 6b, increased elution peak tailing and larger 100 mM acetic acid strip peaks were observed with incorporation of all modifiers tested during elution of bsAb 1 and 3 from Lambda XP. The peak broadening observed with incorporation of the modifiers was not related to pH transitions and suggests an impact to bsAb binding strength to the LC affinity media. Surprisingly, elution with 20 mM of any modifier tested provided near total clearance of mis-paired species, and substantial removal of aggregate and low molecular weight (LMW) impurities for both bsAb 1 and bsAb 3, Table 5. Lambda half antibody was also unexpectedly enriched in the strip peak, whereas in pH gradient screening experiments bsAb 1 half antibody was not found to substantially co-elute with lambda-lambda mis-pairs, Table 6. Additionally, it was observed that the same avidity-binding strengthening with a 20 mM NaCl, pH 3.5 elution method on LambdaFabSelect media for bsAb 1, but with less impurity clearance compared to Lambda XP, Table 6 in the supplementary material.

TABLE 5
Lambda XP Purification Process Performance with Elution Modifiers

| | | | | Purity | | | |
|---|---|---|---|---|---|---|---|
| | | | | by | | | |
| | | Step | Monomer | HIC- | Elution | | Product |
| Elution | bsAb | yield | by HP- | HPLC | volume | Product | conductivity |
| Condition | material | (%) | SEC (%) | (%) | (CV) | pH | (mS/cm) |
| No | bsAb 1 | 86 | 88.5 | 94.9 | 2.1 | 4.0 | 0.3 |
| modifier | | | | | | | |
| NaCl | bsAb 1 | 73 | 95.0 | 98.3 | 3.9 | 3.9 | 2.6 |
| MgCl₂ | bsAb 1 | 70 | 96.1 | 98.8 | 5.1 | 3.5 | 4.6 |
| Arginine | bsAb 1 | 75 | 94.8 | 98.1 | 3.9 | 3.8 | 2.2 |
| No | bsAb 3 | 79 | 89.7 | 84.2 | 3.5 | —^{a} | —^{a} |
| modifier | | | | | | | |
| NaCl | bsAb 3 | 57 | 97.0 | 98.8 | 4.5 | —^{a} | —^{a} |
| MgCl₂ | bsAb 3 | 52 | 97.3 | 98.9 | 4.5 | —^{a} | —^{a} |
| Arginine | bsAb 3 | 44 | 96.9 | 99.0 | 5.8 | —^{a} | —^{a} |

^{a}Not tested

TABLE 6
Lambda LC affinity with 20 mM NaCl, pH 3.5 step elution product quality

| | | | Purity | | Half | LC | Free |
|---|---|---|---|---|---|---|---|
| | | | by HIC- | Aggregate | antibody | dimer | LC by |
| LC affinity | bsAb | | HPLC | by HPSEC | by NR- | by NR- | NR-CGE |
| media | construct | Fraction | (%) | (%) | CGE (%) | CGE (%) | (%) |
| CaptureSelect | bsAb 1 | Load | 86.8 | 13.5 | 1.4 | 1.3 | 1 |
| Lambda XP | | Elution | 98.3 | 3.4 | 0.2 | 0 | 0.5 |
| | | Strip | —^{a} | 43.2 | 13.4 | 29.3 | 4.8 |
| LambdaFabSelect | bsAb 1 | Load | 86.8 | 13.5 | 1.4 | 1.3 | 1 |
| | | Elution | 97.0 | 6.1 | 0.1 | 0 | 0.4 |
| | | Strip | 53.1 | 34.3 | —^{a} | —^{a} | —^{a} |

^{a}Not tested

For protein A media, Tustian et al found that chaotropic salts promote bsAb heterodimer product elution at higher pH (A. D. Tustian, et al., MAbs. 8 (2016) 828-838). For CaptureSelect Kappa XP, there was no clear trend with respect to chaotropic nature of the modifiers, and the highly chaotropic salt MgCl₂ actually produced bsAb heterodimer elution at a lower pH than the control condition, Table 4. In comparison, Chen et al found that for protein L affinity media, addition of salts during elution instead preferentially increased the binding strength of their mis-paired bsAb (C. Chen, et al., MAbs. 11 (2019) 632-638).

For CaptureSelect Lambda XP, it is unintuitive that just 20 mM NaCl would substantially impact binding strength between protein and affinity ligand. Compared to previous data for protein L media showing 50-100 mM NaCl adequate for enhancing binding-valency mediated separations, CaptureSelect Lambda XP requires even less salt at 20 mM. Similar to CaptureSelect Kappa XP and prior data for protein L, binding valency-mediated separation on CaptureSelect Lambda XP appears to be enhanced by preferential strengthening of impurity binding to the media with addition of elution modifiers. However, binding strength of the bsAbs and impurities are much more sensitive to salt addition/conductivity on CaptureSelect Lambda XP compared to CaptureSelect Kappa XP.

Significantly, these elution methods for Lambda LC affinity chromatography media are highly amenable to scale-up for large scale biotherapeutic manufacturing. The low concentrations of familiar elution modifiers required for increased impurity separation will have minimal impact on subsequent purification or virus inactivation unit operations. For practical scale-up of this method, a strategy can be designed around load challenge, elution pH, and salt concentration. Elution peak tailing may also notably produce scale-up challenges due to limitations of intermediate product holding tanks, but strategies around peak collection criteria and further mobile phase condition optimization may alleviate this issue.

LC affinity chromatography is cementing as a robust platform for asymmetric bsAb purification. Existing and emerging commercial LC affinity media provide attractive properties allowing productive processes for large scale antibody therapeutic manufacture. The consistent capability of commercial light chain affinity media to separate mis-paired variants, aggregate, and LMW impurities of model asymmetric bsAbs by leveraging binding avidity was demonstrated. Through pH gradient screening, CaptureSelect Kappa XP and Lambda XP was found to provide greatest baseline resolution of product variants possessing more than one light chain of the same class as the media specificity. Differences in resolution across the different chromatography media tested are not adequately explained only by properties of the media base matrix. It appears that these effects may also be dependent on ligand and/or attachment chemistry specific attributes. Specific running conditions of our experiments such as the residence time, protein load, and gradient slope also played a role in the resolutions achieved. Half antibody and free light chain impurities from some of bsAb constructs exhibited increased binding strength compared to bsAb heterodimer on respective LC affinity media. These findings may suggest that avidity-binding alone does not fully explain observed increases to binding strength on affinity media.

These separations can be enhanced on CaptureSelect Kappa XP and Lambda XP chromatography media by addition of elution modifiers. CaptureSelect Kappa XP media requires much higher concentrations of elution modifiers to provide substantial increases to impurity resolution compared to CaptureSelect Lambda XP media. Addition of 500 mM magnesium chloride surprisingly provided baseline separation on CaptureSelect Kappa XP media, with mis-paired and half antibody species still bound at pH 2.8. For CaptureSelect Lambda XP media, addition of just 20 mM NaCl provided substantial separation of bsAb heterodimer from relevant impurities with an isocratic elution condition. For both CaptureSelect Kappa XP and Lambda XP, results suggest that improved separation is achieved through preferential strengthening of impurity binding. Comparing these findings to previous literature, some similarities to protein A and protein L binding avidity separations can be found. However, previous conclusions on trends of the chaotropic nature of modifiers or disruption of repulsive forces do not appear to fully explain the phenomenon observed with CaptureSelect Kappa XP and Lambda XP media, and additional work needs to be done to elucidate the mechanisms.

Scalability of these processes may be improved by new chromatography media that provide further enhanced mass transfer properties and perhaps more importantly with continued ligand optimization. Further ligand optimization such as increased ligand densities, increased flexibility, lower impurity binding, and other changes may provide increased binding capacity and greater accessibility for multi-binding events. Deeper investigation into the mechanisms by which lyotropic salts or other modifiers affect binding strength of multivalent ligand-protein interactions may expand upon the operating spaces to optimize these separations for different affinity media. Evaluation of potential differences in binding and dissociation kinetics, binding orientations, and protein conformations may also provide additional insight.

What is claimed is:

1. A method of purifying an asymmetric bispecific antibody comprising: (a) contacting a composition comprising the bispecific antibody with a lambda light chain affinity matrix; (b) washing the affinity matrix to remove impurities; and (c) eluting the bispecific antibody with an elution buffer comprising between about 5 mM to about 45 mM of a salt.

2. The method of claim 1, wherein the elution buffer further comprises an aggregation inhibitor.

3. The method of claim 2, wherein the aggregation inhibitor is arginine-HCl.

4. The method of any one of claims 1-3, wherein the concentration of the salt is about 20 mM.

5. The method of any one of claims 1-4, wherein the salt is sodium chloride, magnesium chloride, ammonium chloride, sodium acetate, sodium citrate, sodium phosphate, sodium sulfate, arginine-HCl, or histidine-HCl.

6. The method of any one of claims 1-5, wherein the pH of the elution buffer is between about 3 and about 4.5.

7. The method of claim 6, wherein the pH of the elution buffer is about 3.5.

8. The method of any one of claims 1-7, further comprising contacting the asymmetric bispecific antibody with a kappa light chain affinity matrix and eluting the bound bispecific antibody.

9. The method of claim 8, wherein contacting the asymmetric bispecific antibody to a kappa light chain affinity matrix is performed prior to contacting the bispecific antibody with a lambda light chain affinity matrix.

10. The method of claim 8 or 9, wherein the eluent obtained following elution is directly passed over the second affinity matrix.

11. The method of any one of claims 1-10, wherein the method is run in a closed system.

12. The method of any one of claims 1-11, wherein the kappa light chain affinity matrix and/or the lambda light chain affinity matrix is coupled to a solid support.

13. The method of claim 12, wherein the solid support is cross-linked agarose.

14. The method of any one of claims 1-13, wherein the bispecific antibody comprises a modified heavy chain and/or a modified light chain.

15. The method of claim 14, wherein the bispecific antibody comprises: (a) a Fab region comprising a modified heavy chain, wherein the CH1 region of the modified heavy chain comprises (i) a substitution of a native cysteine to a non-cysteine amino acid, and (ii) a substitution of a native non-cysteine amino acid to a cysteine amino acid; (b) a modified corresponding light chain, wherein the CL region of the modified light chain comprises (i) a substitution of a native cysteine to a non-cysteine amino acid, and (ii) a substitution of a native non-cysteine amino acid to a cysteine amino acid; (c) a second Fab region comprising a second heavy chain; and (d) a second corresponding light chain, wherein the modified heavy chain is directly linked to the corresponding modified light chain, and on a separate target binding arm, the second heavy chain is directly linked to the second corresponding light chain, and wherein the substituted cysteine of the modified heavy chain, resulting from the substitution of the native non-cysteine amino acid to the cysteine amino acid, and the substituted cysteine of the modified corresponding light chain, resulting from the substitution of the native non-cysteine amino acid to the cysteine amino acid, can form a disulfide bond.

16. The method of claim 15, wherein (a) the second heavy chain and second corresponding light chain do not comprise a substitution of a native non-cysteine amino acid to a cysteine amino acid and do not comprise a substitution of a native cysteine to a non-cysteine amino acid; and/or (b) the two light chains each comprise a VL domain and a CL domain, wherein the VL domains have different amino acid sequences and the CL domains have different amino acid sequences; and/or (c) the two heavy chains each comprise a VH domain, a CH1 domain and an Fc region, wherein the VH domains have different amino acid sequences, the CH1 domains have different amino acid sequences, and the Fab regions have different amino acid sequences, optionally wherein one light chain is a kappa light chain and one light chain is a lambda light chain.

17. The method of claim 16, wherein the two heavy chains form a heterodimer.

18. The method of any one of claims 14-17, wherein the bispecific antibody specifically binds to two independent antigens or to two independent epitopes on the same antigen.

19. The method of any one of claims 15-18, wherein the Fc region of either or both heavy chains comprises one or more modifications, optionally wherein the modifications facilitate heterodimerization of the heavy chains.

20. The method of any one of claims 1-19, wherein the method results in the clearance of mis-paired species, removal of aggregates, and/or removal of low molecular weight impurities from the composition.
